(19) United States
(12) Patent Application Publication
(10) Pub. No.: US 2024/0202916 A1
(43) Pub. Date: Jun. 20, 2024

(54) OBJECT DETECTION APPARATUS, LEARNING APPARATUS, LEARNING METHOD, OBJECT DETECTION PROGRAM, AND STORAGE MEDIUM

(71) Applicant: NEC Corporation, Tokyo (JP)

(72) Inventor: Azusa SAWADA, Tokyo (JP)

(73) Assignee: NEC Corporation, Tokyo (JP)

(21) Appl. No.: 18/417,144

(22) Filed: Jan. 19, 2024

(30) Foreign Application Priority Data
Jun. 13, 2022 (WO) PCT/JP2022/023572

(51) Int. Cl. G06T 7/00; G16H 30/20; G16H 30/40

(52) U.S. Cl. CPC G06T 7/0012; G16H 30/20; G16H 30/40; G06T 2207/10068; G06T 2207/20081; G06T 2207/30096

(57) ABSTRACT

In order to achieve object detection with high accuracy by additionally using an image such as a background image in accordance with a situation, an object detection apparatus (1) includes: an image acquisition section (11) that acquires a first image; a calculation section (12) that uses a first model to calculate a first map from the first image; and a detection section (13) that carries out object detection with reference to at least the first map, in a case where the image acquisition section (11) acquires not only the first image but also a second image, the calculation section (12) using a second model to calculate a second map from the second image or from the first image and the second image, and the detection section (13) carrying out object detection with reference to not only the first map but also the second map.

This application is a Continuation of U.S. application Ser. No. 18/289,304, filed Nov. 2, 2023, which is a National Stage Entry of PCT/JP2023/021720 filed on Jun. 12, 2023, which claims priority from Japanese Patent Application PCT/JP2022/023572 filed on Jun. 13, 2022, the contents of all of which are incorporated herein by reference, in their entirety.

### TECHNICAL FIELD

The present invention relates to a technique for detecting an object from an image.

### BACKGROUND ART

A technique for detecting an object from an image is known. In object detection, an improvement in detection accuracy by difference information can be expected in a case where a background image (for example, in the case of absence of a target object) can be used in combination in addition to a main image. For example, Patent Literature 1 discloses using (i) an input image including an object and (ii) a background image to detect a position of the object. Non-Patent Literatures 1 and 2 each propose a learning method (privileged learning) in which a depth image is used as additional information.

### CITATION LIST

### Patent Literature

### [Patent Literature 1]

Japanese Patent Application Publication Tokukai No. 2017-191501

### Non-Patent Literature

### [Non-Patent Literature 1]

Judy Hoffman et al., “Learning with Side Information through Modality Hallucination” in CVPR 2016

### [Non-Patent Literature 2]

Shanxin Yuan et al., “3D Hand Pose Estimation from RGB Using Privileged Learning with Depth Data” in ICCV 2019

### SUMMARY OF INVENTION

### Technical Problem

However, the technique disclosed in Patent Literature 1 has a problem of (i) always requiring a background image in order to carry out reasoning and (ii) preventing reasoning from being carried out in a situation where a background image cannot be obtained, for example, in a case where object detection is carried out at a new photographing location. The techniques disclosed in Non-Patent Literatures 1 and 2 have, on the contrary, a problem of preventing a background image from being practically used even in a case where the background image is present during reasoning.

An example aspect of the present invention has been made in view of the above problems, and an example object thereof is to achieve object detection with high accuracy by additionally using an image such as a background image in accordance with a situation.

### Solution to Problem

An object detection apparatus according to an example aspect of the present invention includes at least one processor, the at least one processor carrying out: an image acquisition process for acquiring a first image; a calculation process for using a first model to calculate a first map from the first image; and a detection process for carrying out object detection with reference to at least the first map, in a case where the at least one processor acquires not only the first image but also a second image in the image acquisition process, in the calculation process, the at least one processor using a second model to calculate a second map from the second image or from the first image and the second image, and in the detection process, the at least one processor carrying out object detection with reference to not only the first map but also the second map.

A learning apparatus according to an example aspect of the present invention includes at least one processor, the at least one processor carrying out: a training data acquisition process for acquiring training data which includes at least one first image, at least one second image, and label information indicative of an object included in the at least one first image; a first learning process for training a first model with reference to the at least one first image and the label information which are included in the training data, the first model calculating a first map from a first image; and a second learning process for training the first model and a second model with reference to the at least one first image, the at least one second image, and the label information which are included in the training data, the second model calculating a second map from a second image.

A learning method according to an example aspect of the present invention includes: acquiring training data which includes at least one first image, at least one second image, and label information indicative of an object included in the at least one first image; training a first model with reference to the at least one first image and the label information which are included in the training data, the first model calculating a first map from a first image; and training the first model and a second model with reference to the at least one first image, the at least one second image, and the label information which are included in the training data, the second model calculating a second map from a second image.

An example aspect of the present invention makes it possible to achieve object detection with high accuracy by additionally using an image such as a background image in accordance with a situation.

### BRIEF DESCRIPTION OF DRAWINGS

FIG. 1 is a block diagram illustrating a configuration of an object detection apparatus according to a first example embodiment.

FIG. 2 is a flowchart showing a flow of an object detection method according to the first example embodiment.

FIG. 3 is a block diagram illustrating a configuration of a learning apparatus according to the first example embodiment.

FIG. 4 is a flowchart showing a flow of a learning method according to the first example embodiment.

FIG. 5 is a block diagram illustrating a configuration of an information processing apparatus according to a second example embodiment.

FIG. 6 is a diagram illustrating an overview of an object detection process according to the second example embodiment.

FIG. 7 is a diagram illustrating a specific example of the object detection process according to the second example embodiment.

FIG. 8 is a flowchart showing a flow of an object detection method according to the second example embodiment.

FIG. 9 is a block diagram illustrating a configuration of an information processing apparatus according to a third example embodiment.

FIG. 10 is a block diagram illustrating example hardware configurations of an object detection apparatus, a learning apparatus, and an information processing apparatus according to the example embodiments.

### DESCRIPTION OF EMBODIMENTS

### First Example Embodiment

The following description will discuss a first example embodiment of the present invention in detail with reference to the drawings. The present example embodiment is an embodiment serving as a basis for example embodiments described later.

### (Configuration of Object Detection Apparatus)

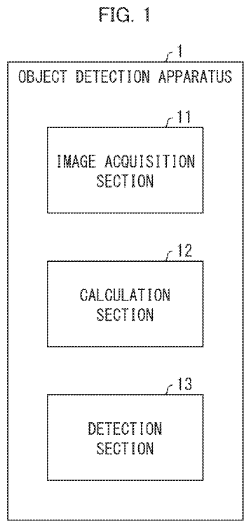

The following description will discuss a configuration of an object detection apparatus 1 according to the present example embodiment with reference to FIG. 1. FIG. 1 is a block diagram illustrating the configuration of the object detection apparatus 1. The object detection apparatus 1 includes an image acquisition section 11, a calculation section 12, and a detection section 13.

The image acquisition section 11 acquires a first image. The calculation section 12 uses a first model to calculate a first map from the first image. The detection section 13 carries out object detection with reference to at least the first map.

In a case where the image acquisition section 11 acquires not only the first image but also a second image, the calculation section 12 uses a second model to calculate a second map from the second image or from the first image and the second image, and the detection section 13 carries out object detection with reference to not only the first map but also the second map.

As described above, a configuration is employed such that the object detection apparatus 1 according to the present example embodiment includes: the image acquisition section 11 that acquires a first image; the calculation section 12 that uses a first model to calculate a first map from the first image; and the detection section 13 that carries out object detection with reference to at least the first map, in a case where the image acquisition section 11 acquires not only the first image but also a second image, the calculation section 12 using a second model to calculate a second map from the second image or from the first image and the second image, and the detection section 13 carrying out object detection with reference to not only the first map but also the second map. Thus, the object detection apparatus 1 according to the present example embodiment brings about an effect of making it possible to achieve object detection with high accuracy by additionally using an image such as a background image in accordance with a situation.

### (Flow of Object Detection Method)

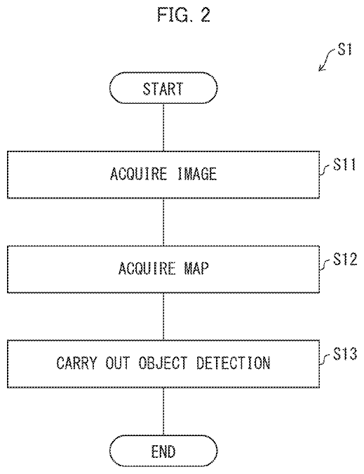

The following description will discuss a flow of an object detection method S1 according to the present example embodiment with reference to FIG. 2. FIG. 2 is a flowchart showing the flow of the object detection method S1. Note that steps of the object detection method S1 may be carried out by a processor of the object detection apparatus 1 or by a processor of another apparatus. Alternatively, the steps may be carried out by processors provided in respective different apparatuses.

In a step S11, at least one processor acquires a first image. In a step S12, the at least one processor uses a first model to calculate a first map from the first image. In a step S13, the at least one processor carries out object detection with reference to at least the first map.

In a case where not only the first image but also a second image is acquired, in the step 12, the at least one processor uses a second model to calculate a second map from the second image or from the first image and the second image, and in the step 13, the at least one processor carries out object detection with reference to not only the first map but also the second map.

As described above, a configuration is employed such that the object detection method S1 according to the second example embodiment includes: (a) acquiring a first image; (b) using a first model to calculate a first map from the first image; and (c) carrying out object detection with reference to at least the first map, in a case where not only the first image but also a second image is acquired, in (b), a second model being used to calculate a second map from the second image or from the first image and the second image, and in (c), object detection being carried out with reference to not only the first map but also the second map. Thus, the object detection method S1 according to the present example embodiment brings about an effect of making it possible to achieve object detection with high accuracy by additionally using an image such as a background image in accordance with a situation.

### (Configuration of Learning Apparatus)

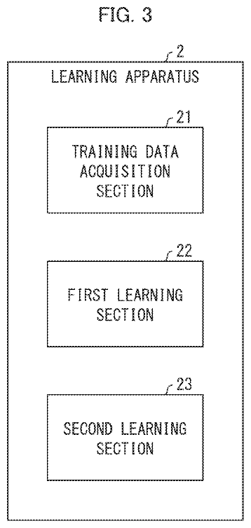

The following description will discuss a configuration of a learning apparatus 2 according to the present example embodiment with reference to FIG. 3. FIG. 3 is a block diagram illustrating the configuration of the learning apparatus 2. The learning apparatus 2 includes a training data acquisition section 21, a first learning section 22, and a second learning section 23.

The training data acquisition section 21 acquires training data which includes at least one first image, at least one second image, and label information indicative of an object included in the at least one first image. The first learning section 22 trains a first model with reference to the at least one first image and the label information which are included in the training data, the first model calculating a first map from a first image. The second learning section 23 trains the first model and a second model with reference to the at least one first image, the at least one second image, and the label information which are included in the training data, the second model calculating a second map from a second image.

As described above, a configuration is employed such that the learning apparatus 2 according to the present example embodiment includes: the training data acquisition section 21 that acquires training data which includes at least one first image, at least one second image, and label information indicative of an object included in the at least one first image; the first learning section 22 that trains a first model with reference to the at least one first image and the label information which are included in the training data, the first model calculating a first map from a first image; and the second learning section 23 that trains the first model and a second model with reference to the at least one first image, the at least one second image, and the label information which are included in the training data, the second model calculating a second map from a second image. Thus, the learning apparatus 2 according to the present example embodiment brings about an effect of making it possible to provide a model that achieves object detection with high accuracy by additionally using an image such as a background image in accordance with a situation.

### (Flow of Learning Method)

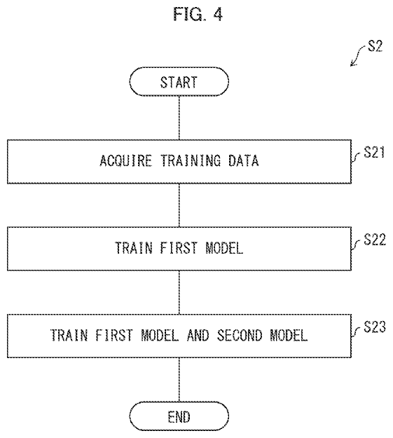

The following description will discuss a flow of a learning method S2 according to the present example embodiment with reference to FIG. 4. FIG. 4 is a flowchart showing the flow of the learning method S2. Note that steps of the learning method S2 may be carried out by a processor of the learning apparatus 2 or by a processor of another apparatus. Alternatively, the steps may be carried out by processors provided in respective different apparatuses.

In a step S21, at least one processor acquires training data which includes at least one first image, at least one second image, and label information indicative of an object included in the at least one first image. In a step S22, the at least one processor trains a first model with reference to the at least one first image and the label information which are included in the training data, the first model calculating a first map from a first image. In a step S23, the at least one processor trains the first model and a second model with reference to the at least one first image, the at least one second image, and the label information which are included in the training data, the second model calculating a second map from a second image.

As described above, the learning method S2 according to the present example embodiment includes: acquiring training data which includes at least one first image, at least one second image, and label information indicative of an object included in the at least one first image; training a first model with reference to the at least one first image and the label information which are included in the training data, the first model calculating a first map from a first image; and training the first model and a second model with reference to the at least one first image, the at least one second image, and the label information which are included in the training data, the second model calculating a second map from a second image. Thus, the learning method S2 according to the present example embodiment brings about an effect of making it possible to provide a model that makes it possible to achieve object detection with high accuracy by additionally using an image such as a background image in accordance with a situation.

### Second Example Embodiment

The following description will discuss a second example embodiment of the present invention in detail with reference to the drawings. Note that members having functions identical to those of the respective members described in the first example embodiment are given respective identical reference numerals, and a description of those members is omitted as appropriate.

### <Configuration of Information Processing Apparatus>

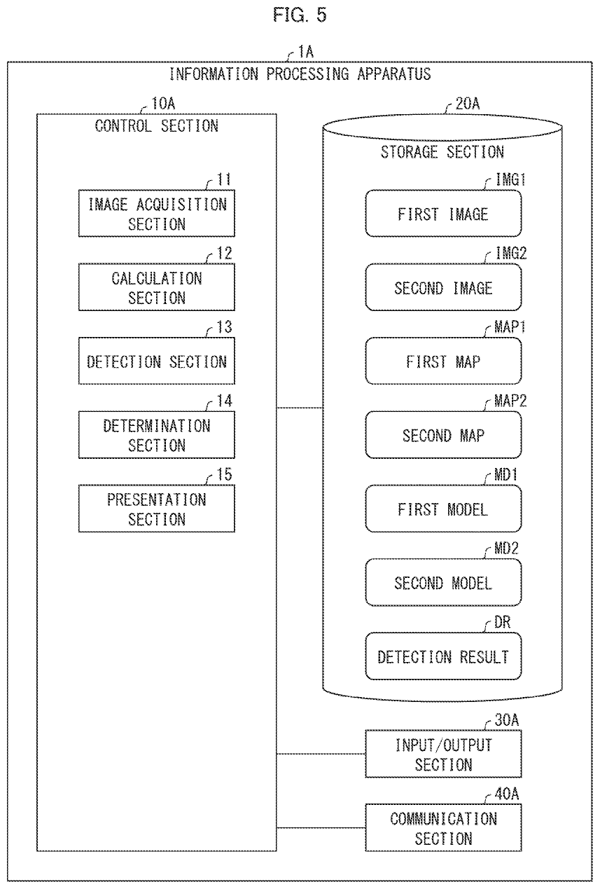

FIG. 5 is a block diagram illustrating a configuration of an information processing apparatus 1A according to the second example embodiment. The information processing apparatus 1A is an apparatus that detects an object from an image. Note here that the object is, for example, a mobile object (e.g., a vehicle or a person) included in a satellite image. Note, however, that the object is not limited to the example described above.

The information processing apparatus 1A includes a control section 10A, a storage section 20A, an input/output section 30A, and a communication section 40A.

### (Input/Output Section)

To the input/output section 30A, an input/output apparatus(es) such as a keyboard, a mouse, a display, a printer, and/or a touch panel is/are connected. The input/output section 30A receives, from an input apparatus(es) connected thereto, an input of various pieces of information to the information processing apparatus 1A. The input/output section 30A outputs, to an output apparatus(es) connected thereto, various pieces of information under control by the control section 10A. Examples of the input/output section 30A include an interface such as a universal serial bus (USB). The input/output section 30A may include, for example, a display panel, a loudspeaker, a keyboard, a mouse, and/or a touch panel.

### (Communication Section)

The communication section 40A communicates, via a communication line, with an apparatus external to the information processing apparatus 1A. A specific configuration of the communication line is not limited to the present example embodiment. Examples of the communication line include a wireless local area network (LAN), a wired LAN, a wide area network (WAN), a public network, a mobile data communication network, and a combination thereof. The communication section 40A transmits, to another apparatus, data supplied from the control section 10A, and supplies, to the control section 10A, data received from another apparatus.

### (Control Section)

The control section 10A includes an image acquisition section 11, a calculation section 12, a detection section 13, a determination section 14, and a presentation section 15.

### (Image Acquisition Section)

The image acquisition section 11 acquires a first image IMG1 or acquires the first image IMG1 and a second image IMG2. The first image IMG1 is to be subjected to an object detection process, and is, for example, an image obtained by photographing an object. The object is, for example, a mobile object (e.g., a vehicle or a person). Note, however, that the object is not limited to such a mobile object. The first image IMG1 includes, for example, an RGB-channel image. Note, however, that the first image IMG1 is not limited to the example described above. Alternatively, the first image IMG1 may be another image.

The second image IMG2 is an image for use in the object detection process, and is, for example, a background image corresponding to the first image IMG1, a depth image sensed by a depth sensor, or an infrared image captured by an infrared camera. Note, however, that the second image IMG2 is not limited to the example described above. Alternatively, the second image IMG2 may be another image.

### (Calculation Section)

The calculation section 12 uses a first model MD1 to calculate a first map MAP1 from the first image IMG1. Note here that the first model MD1 is a model which uses the first image IMG1 as an input and outputs the first map MAP1. The first model MD1 is, for example, a convolutional neural network. The first map MAP1 is a map that is calculated from the first image IMG1. The first map MAP1 is, for example, a feature map that is obtained through a process such as convolution with respect to the first image IMG1. The first map that is calculated by the calculation section 12 is referred to in the object detection process.

In a case where the image acquisition section 11 acquires not only the first image IMG1 but also the second image IMG2, the calculation section 12 uses a second model MD2 to calculate a second map MAP2 from the second image IMG2 or from the first image IMG1 and the second image IMG2. The second model MD2 is a model that outputs the second map MAP2. The second model MD2 is, for example, a convolutional neural network. Note here that an input to the second model MD2 includes, for example, the second image IMG2, or the first image IMG1 and the second image IMG2. The second map MAP2 is a map that is calculated from the second image IMG2 or from the first image and the second image. The second map MAP2 is, for example, a feature map indicating a feature of the second image, or a weight map indicating a difference between the second image IMG2 and the first image IMG1.

### (Detection Section)

The detection section 13 carries out object detection with reference to at least the first map MAP1. For example, the detection section 13 carries out object detection by a method of object detection such as Faster Regions with CNN features (R-CNN), Single Shot MultiBox Detector (SSD), or You Only Look Once (YOLO). Note here that the detection section 13 may be configured as a model of, for example, a subsequent stage (R-CNN) of Faster R-CNN. Alternatively, the calculation section 12 and the detection section 13 that are connected with each other may be configured as a model of, for example, a preceding stage (Region Proposal Networks (RPN)) of Faster R-CNN, SSD, or YOLO. Note, however, that the method by which the detection section 13 carries out object detection is not limited to the example described above. Alternatively, the detection section 13 may carry out object detection by another method.

In a case where the image acquisition section 11 acquires not only the first image IMG1 but also the second image IMG2, the detection section 13 carries out object detection with reference to not only the first map MAP1 but also the second map MAP2. For example, the detection section 13 carries out object detection with reference to a third map that is obtained through a computation with use of the first map MAP1 and the second map MAP2.

The third map is a map that is obtained through a computation with use of the first map MAP1 and the second map MAP2. The third map is, for example, a map that is obtained by multiplying the first map MAP1 by the second map MAP2. In this case, in other words, in a case where the image acquisition section 11 acquires not only the first image IMG1 but also the second image IMG2, the detection section 13 carries out object detection with reference to the third map that is obtained by multiplying the first map MAP1 by the second map MAP2. Note, however, that the third map is not limited to the example described above. Alternatively, the third map may be a map that is obtained through another computation. The third map may be, for example, a map that is obtained by adding the second map MAP2 to the first map MAP1.

### (Determination Section)

The determination section 14 carries out a determination process for determining whether the image acquisition section 11 acquires the first image IMG1 or acquires the first image IMG1 and the second image IMG2. For example, the determination section 14 carries out the determination process with reference to a flag indicating whether the first image IMG1 is acquired or whether the first image IMG1 and the second image IMG2 are acquired. Note, however, that the determination process carried out by the determination section 14 is not limited to the example described above. Alternatively, the determination section 14 may carry out the determination process by another method.

### (Presentation Section)

The presentation section 15 presents a result of object detection by the detection section 13. The presentation section 15 may present the result by outputting the result to an output apparatus(es) (a display, a loudspeaker, a printer, and/or the like) connected to the input/output section 30A. Alternatively, the presentation section 15 may transmit the result to another apparatus connected via the communication section 40A. For example, the presentation section 15 displays, on a display panel of the input/output section 30A, an image showing the result of object detection.

### (Storage Section)

The storage section 20A stores the first image IMG1, the second image IMG2, the first map MAP1, the second map MAP2, the first model MD1, the second model MD2, and a detection result DR.

### <Overview of Object Detection Process>

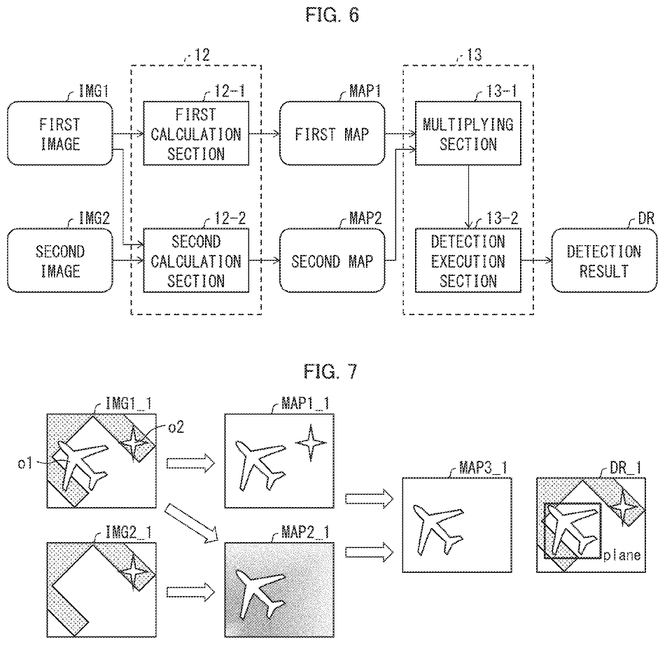

FIG. 6 is a diagram illustrating an example overview of an object detection process that is carried out by the information processing apparatus 1A. In the example of FIG. 6, the calculation section 12 includes a first calculation section 12-1 and a second calculation section 12-2. The first calculation section 12-1 uses the first model MD1 to calculate the first map MAP1 from the first image IMG1. The second calculation section 12-2 uses the second model MD2 to calculate the second map MAP2 from the second image IMG2 or from the first image IMG1 and the second image IMG2. The second map MAP2 is, for example, a weight map indicating a difference between the first image IMG1 and the second image IMG2. In a case where the second image IMG2 is not acquired, the calculation section 12 does not carry out a process for calculating the second map MAP2.

The detection section 13 includes a multiplying section 13-1 and a detection execution section 13-2. The multiplying section 13-1 calculates the third map by multiplying the first map MAP1 by the second map MAP2. The multiplying section 13-1 may apply a multiplication process to all of first maps MAP1, or may apply the multiplication process to some of the first maps MAP1.

In a case where the image acquisition section 11 acquires the second image IMG2, the detection execution section 13-2 carries out object detection with reference to the third map. In contrast, in a case where the image acquisition section 11 does not acquire the second image IMG2, the detection execution section 13-2 carries out object detection with reference to the first map MAP1.

For example, the detection execution section 13-2 detects an object on the basis of an output that is obtained by inputting the feature map (first map MAP1 or third map) to a trained model. Note here that the trained model is, for example, a model constructed by supervised machine learning. The trained model is, for example, a convolutional neural network. An input to the trained model includes, for example, a feature map of a candidate region, and an output from the trained model includes, for example, a type of the object and information indicative of a circumscribed rectangle of the object. Examples of a method by which the detection execution section 13-2 detects the object from the feature map include the above-described methods such as Faster R-CNN and SSD.

### <Specific Example of Object Detection Process>

FIG. 7 is a diagram illustrating a specific example of the object detection process according to the second example embodiment. In the example of FIG. 7, a main image IMG1_1 is an example of the first image IMG1, and an additional image IMG2_1 is an example of the second image IMG2. In the example of FIG. 7, the image acquisition section 11 acquires the main image IMG1_1 and the additional image IMG2_1. The main image IMG1_1 is an image of a candidate region extracted by RPN (described earlier). The additional image IMG2_1 is a background image of the candidate region. The main image IMG1_1 is a part of an image obtained by photographing an object. The additional image IMG2_1 is a part of a captured image that corresponds to the main image IMG1_1 image and that does not include the object.

The main image IMG1_1 includes an object o1 and an object o2. The object o1 is an object to be detected. In contrast, the object o2 is an object that is included also in the additional image IMG2_1 and that does not need to be detected. Thus, a feature map MAP1_1 includes the object o2 that is different from the object o1 to be detected and that is incorrect attention.

The calculation section 12 calculates the feature map MAP1_1 by inputting the main image IMG1_1 to the first model MD1. The feature map MAP1_1 is an example of the first map MAP1. The calculation section 12 also calculates a weight map MAP2_1 by inputting the main image IMG1_1 and the additional image IMG2_1 to the second model MD2. The weight map MAP2_1 is an example of the second map MAP2. Note here that since the object o2 is included in both the main image IMG1_1 and the additional image IMG2_1, the object o2 does not or is less likely to appear in the weight map MAP2_1 indicating a difference between the main image IMG1_1 and the additional image IMG2_1.

The detection section 13 calculates a feature map MAP3_1 by multiplying the feature map MAP1_1 by the weight map MAP2_1. The feature map MAP3_1 is an example of the third map. By multiplying the feature map MAP1_1 by the weight map MAP2_1, the object o2 included in the feature map MAP1_1 does not or is less likely to appear in the feature map MAP3_1.

The detection section 13 calculates a detection result DR_1 for the object (a result of reestimation of a type of the object and a circumscribed rectangle of the object) with reference to the feature map MAP3_1. For example, the detection result DR_1 is presented by the presentation section 15.

### <Flow of Object Detection Method>

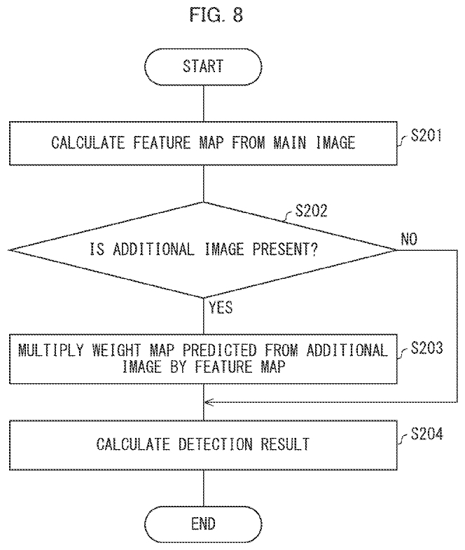

FIG. 8 is a flowchart showing a flow of an example of an object detection method according to the second example embodiment.

### (Step S201)

In a step S201, the calculation section 12 calculates the feature map MAP1_1 from the main image IMG1_1.

### (Step S202)

In a step S202, the determination section 14 determines whether the additional image IMG2_1 is present. For example, the determination section 14 determines, with reference to a predetermined flag (e.g., a flag assigned to the main image IMG1_1), whether the additional image IMG2_1 is present. In a case where the additional image IMG2_1 is present (“YES” in the step S202), the determination section 14 proceeds to the process in a step S203. In contrast, in a case where the additional image IMG2_1 is absent (“NO” in the step S202), the determination section 14 proceeds to the process in a step S204.

### (Step S203)

In the step S203, the detection section 13 multiplies the feature map MAP1_1 by the weight map MAP2_1 calculated from the additional image IMG2_1, and calculates the feature map MAP3_1.

### (Step S204)

In the step S204, the detection section 13 calculates a result of detection of the object from the feature map MAP3_1 calculated in the step S203.

### <Effect of Information Processing Apparatus>

As described above, a configuration is employed such that in the information processing apparatus 1A according to the present example embodiment, in a case where the image acquisition section 11 acquires not only the first image IMG1 but also the second image IMG2, the detection section 13 carries out object detection with reference to the third map obtained by multiplying the first map MAP1 by the second map MAP2. Thus, according to the information processing apparatus 1A according to the present example embodiment, an effect of making it possible to detect an object with higher accuracy is obtained by carrying out object detection with reference to the third map obtained by multiplying the first map MAP1 by the second map MAP2.

Furthermore, a configuration is employed such that the information processing apparatus 1A according to the present example embodiment further includes the determination section 14 that carries out a determination process for determining whether the image acquisition section 11 acquires the first image IMG1 or acquires the first image IMG1 and the second image IMG2. Thus, the information processing apparatus 1A according to the present example embodiment brings about an effect of (i) making it possible to detect an object in both a case where the second image is acquired and a case where the second image is not acquired and (ii) making it possible to detect the object with higher accuracy in a case where the second image is present. More specifically, for example, in a situation where a background image may be obtained in addition to a main image, it is possible to practically use the background image to improve accuracy during reasoning.

Moreover, a configuration is employed such that in the information processing apparatus 1A according to the present example embodiment, the determination section 14 carries out the determination process with reference to a flag indicating whether the first image IMG1 is acquired or whether the first image IMG1 and the second image IMG2 are acquired. Thus, according to the information processing apparatus 1A according to the present example embodiment, determining, with reference to a flag, whether the second image is acquired brings about an effect of (i) making it possible to detect an object in both a case where the second image is acquired and a case where the second image is not acquired and (ii) making it possible to detect the object with higher accuracy in a case where the second image is present.

### Third Example Embodiment

The following description will discuss a third example embodiment of the present invention in detail with reference to the drawings. Note that members having functions identical to those of the respective members described in the first example embodiment are given respective identical reference numerals, and a description of those members is not repeated.

### <Configuration of Information Processing Apparatus>

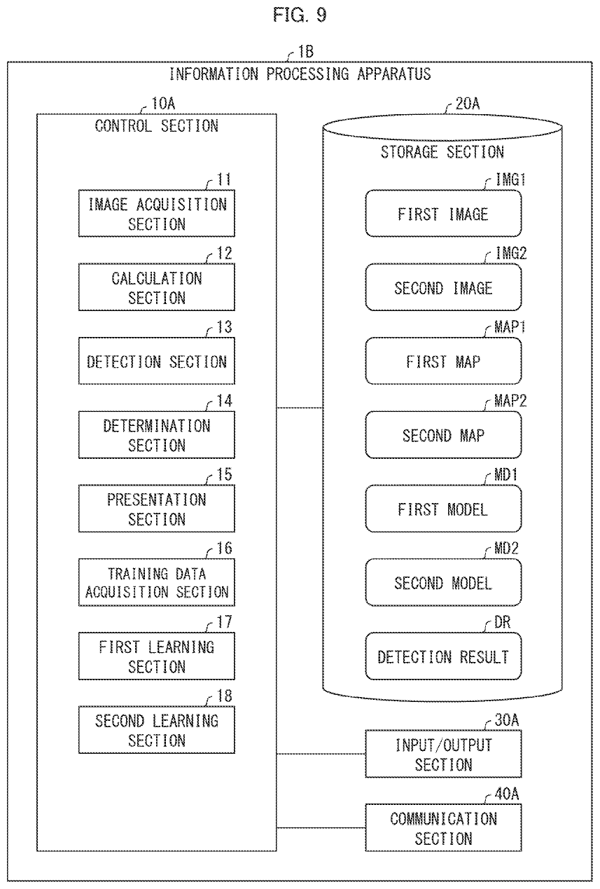

FIG. 9 is a block diagram illustrating a configuration of an information processing apparatus 1B according to the third example embodiment. A control section 10A of the information processing apparatus 1B includes not only an image acquisition section 11, a calculation section 12, a detection section 13, a determination section 14, and a presentation section 15 but also a training data acquisition section 16, a first learning section 17, and a second learning section 18. The training data acquisition section 16, the first learning section 17, and the second learning section 18 constitute a learning apparatus according to the present specification.

### (Training Data Acquisition Section)

The training data acquisition section 16 acquires training data which includes at least one first image, at least one second image, and label information indicative of an object included in the at least one first image. Note here that the first image and the second image are as described in the second example embodiment disclosed above. The label information includes, for example, information indicative of a type of the object.

### (First Learning Section)

The first learning section 17 trains a first model MD1 by machine learning with reference to the at least one first image and the label information which are included in the training data. The first model MD1 is, as described earlier, a model that is used by the calculation section 12 to calculate a first map MAP1. The first model MD1 is, for example, a convolutional neural network. In the present example embodiment, for example, even in a case where the training data includes a second image, the first learning section 17 may train the first model MD1, without using the second image, by supervised machine learning in which a set of the first image and the label information is used.

### (Second Learning Section)

The second learning section 18 trains the first model MD1 and a second model MD2 by machine learning with reference to the at least one first image, the at least one second image, and the label information which are included in the training data. The second model MD2 is, as described earlier, a model that is used by the calculation section 12 to calculate a second map MAP2. The second model MD2 is, for example, a convolutional neural network. In this case, the second learning section 18 may additionally use a loss function that reduces a difference between the first map MAP1 which has not been multiplied by the weight map and the third map MAP3 which has been multiplied by the weight map.

### <Effect of Information Processing Apparatus>

A configuration is employed such that the information processing apparatus 1B according to the present example embodiment includes: the training data acquisition section 16 that acquires training data which includes at least one first image, at least one second image, and label information indicative of an object included in the at least one first image; the first learning section 17 that trains the first model MD1 by machine learning with reference to the at least one first image and the label information which are included in the training data; and the second learning section 18 that trains the first model MD1 and the second model MD2 by machine learning with reference to the at least one first image, the at least one second image, and the label information which are included in the training data. Thus, the information processing apparatus 1B according to the present example embodiment brings about not only the effect brought about by the object detection apparatus 1 according to the first example embodiment but also an effect of making it possible to provide a model that makes it possible to achieve object detection with high accuracy by additionally using an image such as a background image in accordance with a situation.

### Examples

The following description will discuss an Example according to the present disclosure. The present Example is an Example in which the information processing apparatuses 1A and 1B according to the example embodiments described earlier are applied to medical and healthcare fields. In the present Example, the first image IMG1 is an image captured by carrying out an endoscopic examination with respect to a subject. The second image IMG2 is an image captured by a past endoscopic examination in the same subject. The second image IMG2 is an image that is obtained in a case where no lesion has been detected and that is obtained by photographing the same place as that in the first image IMG1.

In the present Example, the detection section 13 detects an object that is a lesion which is detected from an image captured by carrying out an endoscopic examination with respect to a subject. In a case where a past endoscopic examination image (the second image IMG2) of the subject is present, the detection section 13 uses a past endoscopic image to carry out lesion detection. The presentation section 15 presents a result of detection of the lesion to a medical worker.

The medical worker refers to the presented result of detection of the lesion, and, for example, determines a treatment method for the subject. In other words, the presentation section 15 outputs the result of detection of the lesion for supporting decision making by the medical worker. That is, according to the present Example, the information processing apparatuses 1A and 1B make it possible to support decision making by the medical worker.

For example, the presentation section 15 may present, to the medical worker, the treatment method that has been determined on the basis of (i) a model generated by machine learning of a correspondence relationship between the result of detection of the lesion and the treatment method and (ii) the result of detection of the lesion of the subject. A method for determining the treatment method is not limited to the example described above. This enables an information processing apparatus to support decision making by a user.

Furthermore, the present Example brings about an effect of (i) making it possible to detect an object (lesion) in both cases with and without a past endoscopic examination image of a subject and (ii) making it possible to detect the lesion with higher accuracy in the case with the past endoscopic examination image of the subject.

### [Software Implementation Example]

Some or all of functions of the object detection apparatus 1, the information processing apparatuses 1A and 1B, and the learning apparatus 2 (hereinafter referred to as “object detection apparatus 1, etc.”) can be realized by hardware such as an integrated circuit (IC chip) or the like or can be alternatively realized by software.

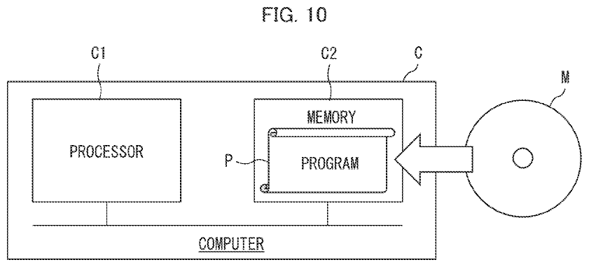

In the latter case, the object detection apparatus 1, etc. are each realized by, for example, a computer that executes instructions of a program that is software realizing the functions. FIG. 10 illustrates an example of such a computer (hereinafter referred to as “computer C”). The computer C includes at least one processor C1 and at least one memory C2. The memory C2 stores a program P for causing the computer C to operate as each of the object detection apparatus 1, etc. In the computer C, the functions of the object detection apparatus 1, etc. are realized by the processor C1 reading the program P from the memory C2 and executing the program P.

The processor C1 may be, for example, a central processing unit (CPU), a graphic processing unit (GPU), a digital signal processor (DSP), a micro processing unit (MPU), a floating point number processing unit (FPU), a physics processing unit (PPU), a microcontroller, or a combination thereof. The memory C2 may be, for example, a flash memory, a hard disk drive (HDD), a solid state drive (SSD), or a combination thereof.

Note that the computer C may further include a random access memory (RAM) in which the program P is loaded when executed and/or in which various kinds of data are temporarily stored. The computer C may further include a communication interface for transmitting and receiving data to and from another apparatus. The computer C may further include an input/output interface for connecting the computer C to an input/output apparatus(es) such as a keyboard, a mouse, a display, and/or a printer.

The program P can also be recorded in a non-transitory tangible storage medium M from which the computer C can read the program P. Such a storage medium M may be, for example, a tape, a disk, a card, a semiconductor memory, a programmable logic circuit, or the like. The computer C can acquire the program P via the storage medium M. The program P can also be transmitted via a transmission medium. The transmission medium may be, for example, a communication network, a broadcast wave, or the like. The computer C can acquire the program P also via the transmission medium.

### ADDITIONAL REMARK 1

The present invention is not limited to the foregoing example embodiments, but may be altered in various ways by a skilled person within the scope of the claims. For example, the present invention also encompasses, in its technical scope, any example embodiment derived by appropriately combining technical means disclosed in the foregoing example embodiments.

### ADDITIONAL REMARK 2

The whole or part of the example embodiments disclosed above can also be described as below. Note, however, that the present invention is not limited to the following supplementary notes.

### (Supplementary Note 1)

An object detection apparatus including: an image acquisition means that acquires a first image; a calculation means that uses a first model to calculate a first map from the first image; and a detection means that carries out object detection with reference to at least the first map, in a case where the image acquisition means acquires not only the first image but also a second image, the calculation means using a second model to calculate a second map from the second image or from the first image and the second image, and the detection means carrying out object detection with reference to not only the first map but also the second map.

### (Supplementary Note 2)

The object detection apparatus according to Supplementary note 1, wherein in a case where the image acquisition means acquires not only the first image but also the second image, the detection means carries out object detection with reference to a third map obtained by multiplying the first map by the second map.

### (Supplementary Note 3)

The object detection apparatus according to Supplementary note 1 or 2, further including a determination means that carries out a determination process for determining whether the image acquisition means acquires the first image or acquires the first image and the second image.

### (Supplementary Note 4)

The object detection apparatus according to Supplementary note 3, wherein the determination means carries out the determination process with reference to a flag indicating whether the first image is acquired or whether the first image and the second image are acquired.

### (Supplementary Note 5)

The object detection apparatus according to Supplementary note 1 or 2, further including: a training data acquisition means that acquires training data which includes at least one first image, at least one second image, and label information indicative of an object included in the at least one first image; a first learning means that trains the first model by machine learning with reference to the at least one first image and the label information which are included in the training data; and a second learning means that trains the first model and the second model by machine learning with reference to the at least one first image, the at least one second image, and the label information which are included in the training data.

### (Supplementary Note 6)

The object detection apparatus according to Supplementary note 1 or 2, further comprising a presentation means that outputs a result of detection by the detection means, the detection means detecting an object that is a lesion which is capable of being detected from an image captured by carrying out an endoscopic examination with respect to a subject, and the presentation means outputting a result of detection of the lesion for supporting decision making by a medical worker.

### (Supplementary Note 7)

A learning apparatus including: a training data acquisition means that acquires training data which includes at least one first image, at least one second image, and label information indicative of an object included in the at least one first image; a first learning means that trains a first model with reference to the at least one first image and the label information which are included in the training data, the first model calculating a first map from a first image; and a second learning means that trains the first model and a second model with reference to the at least one first image, the at least one second image, and the label information which are included in the training data, the second model calculating a second map from a second image.

### (Supplementary Note 8)

An object detection method including: (a) acquiring a first image; (b) using a first model to calculate a first map from the first image; and (c) carrying out object detection with reference to at least the first map, in a case where not only the first image but also a second image is acquired, in (b), a second model being used to calculate a second map from the second image or from the first image and the second image, and in (c), object detection being carried out with reference to not only the first map but also the second map.

### (Supplementary Note 9)

A learning method including: acquiring training data which includes at least one first image, at least one second image, and label information indicative of an object included in the at least one first image; training a first model with reference to the at least one first image and the label information which are included in the training data, the first model calculating a first map from a first image; and training the first model and a second model with reference to the at least one first image, the at least one second image, and the label information which are included in the training data, the second model calculating a second map from a second image.

### (Supplementary Note 10)

An object detection program causing a computer to function as: an image acquisition means that acquires a first image; a calculation means that uses a first model to calculate a first map from the first image; and a detection means that carries out object detection with reference to at least the first map, in a case where the image acquisition means acquires not only the first image but also a second image, the calculation means using a second model to calculate a second map from the second image or from the first image and the second image, and the detection means carrying out object detection with reference to not only the first map but also the second map.

### (Supplementary Note 11)

A learning program causing a computer to function as: a training data acquisition means that acquires training data which includes at least one first image, at least one second image, and label information indicative of an object included in the at least one first image; a first learning means that trains a first model with reference to the at least one first image and the label information which are included in the training data, the first model calculating a first map from a first image; and a second learning means that trains the first model and a second model with reference to the at least one first image, the at least one second image, and the label information which are included in the training data, the second model calculating a second map from a second image.

### ADDITIONAL REMARK 3

The whole or part of the example embodiments disclosed above can also be expressed as follows.

An object detection apparatus including at least one processor, the at least one processor carrying out: an image acquisition process for acquiring a first image; a calculation process for using a first model to calculate a first map from the first image; and a detection process for carrying out object detection with reference to at least the first map, in a case where the at least one processor acquires not only the first image but also a second image in the image acquisition process, in the calculation process, the at least one processor using a second model to calculate a second map from the second image or from the first image and the second image, and in the detection process, the at least one processor carrying out object detection with reference to not only the first map but also the second map.

Note that the object detection apparatus may further include a memory, which may store a program for causing the at least one processor to carry out the image acquisition process, the calculation process, and the detection process. The program may be stored in a non-transitory tangible computer-readable storage medium.

A learning apparatus including at least one processor, the at least one processor carrying out: a training data acquisition process for acquiring training data which includes at least one first image, at least one second image, and label information indicative of an object included in the at least one first image; a first learning process for training a first model with reference to the at least one first image and the label information which are included in the training data, the first model calculating a first map from a first image; and a second learning process for training the first model and a second model with reference to the at least one first image, the at least one second image, and the label information which are included in the training data, the second model calculating a second map from a second image.

Note that the learning apparatus may further include a memory, which may store a program for causing the at least one processor to carry out the training data acquisition process, the first learning process, and the second learning process. The program may be stored in a non-transitory tangible computer-readable storage medium.

### REFERENCE SIGNS LIST

1 Object detection apparatus
1A, 1B Information processing apparatus
2 Learning apparatus
11 Image acquisition section
12 Calculation section
13 Detection section
14 Determination section
15 Presentation section
16, 21 Training data acquisition section
17, 22 First learning section
18, 23 Second learning section

1. A lesion detection apparatus comprising:
a memory storing instructions;
at least one processor configured to execute the instructions to:
acquire one or more images captured by endoscopic examination, the one or more images including a first image;
calculate a first map from the first image with use of a first model;
detect a lesion with reference to at least the first map;
determine whether a second image that is captured by a past endoscopic examination is present, the second image indicating the same place of a same subject as the first image, wherein no lesion is detected from the second image;
in a case where the second image is present, calculate, with use of a second model, a second map from the second image or from both the first image and the second image;
in a case where the second image is present, detect the lesion with reference to both the first map and the second map; and
display a detection result of the lesion to an output device.

2. The lesion detection apparatus according to claim 1, wherein the at least one processor is further configured to execute the instructions to:
in a case where the second image is present, detect the lesion with reference to a third map obtained by multiplying the first map by the second map.

3. The lesion detection apparatus according to claim 1, wherein the determining comprises referring to a flag indicating whether the first image is present or whether the first image and the second image are present.

4. The lesion detection apparatus according to claim 1, wherein the at least one processor is further configured to execute the instructions to:
acquire training data which includes at least one first image, at least one second image, and label information indicative of a lesion included in the at least one first image;
train the first model by machine learning with reference to the at least one first image and the label information which are included in the training data; and
train the first model and the second model by machine learning with reference to the at least one first image, the at least one second image, and the label information which are included in the training data.

5. The lesion detection apparatus according to claim 1, wherein the detection result of the lesion supports decision making by a medical worker.

6. A lesion detection method comprising:
acquiring one or more images captured by endoscopic examination, the one or more images including a first image;
calculating a first map from the first image with use of a first model;
detecting a lesion with reference to at least the first map;
determining whether a second image that is captured by a past endoscopic examination is present, the second image indicating the same place of a same subject as the first image, wherein no lesion is detected from the second image,
in a case where the second image is present, calculating, with use of a second model, a second map from the second image or from both the first image and the second image;
in a case where the second image is present, detecting the lesion with reference to both the first map and the second map; and
displaying a detection result of the lesion to an output device.

7. A non-transitory tangible computer-readable storage medium storing therein a lesion detection program causing a computer to execute the processing comprising:
acquiring one or more images captured by endoscopic examination, the one or more images including a first image;
calculating a first map from the first image with use of a first model;
detecting a lesion with reference to at least the first map;
determining whether a second image that is captured by a past endoscopic examination is present, the second image indicating the same place of a same subject as the first image, wherein no lesion is detected from the second image;
in a case where the second image is present, calculating, with use of a second model, a second map from the second image or from both the first image and the second image;
in a case where the second image is present, detecting the lesion with reference to both the first map and the second map; and
displaying a detection result of the lesion to an output device.
